(19) United States
(12) Patent Application Publication
(10) Pub. No.: US 2006/0045917 A1
(43) Pub. Date: Mar. 2, 2006

(54) Method of conveying matter into targeted cells in a human body

(76) Inventor: Eukki Qi Yu, City of Industry, CA (US)

Correspondence Address: Raymond Y. Chan, 108 N. Ynez Ave., #128, Monterey Park, CA, 91754, US

(21) Appl. No.: 11/212,294

(22) Filed: Aug. 27, 2005

Related U.S. Application Data
(60) Provisional application No. 60/605,678, filed on Aug. 28, 2004.

(51) Int. Cl. C12N 15/87; A61K 9/14

(52) U.S. Cl. 424/489; 435/459

(57) ABSTRACT

A method of conveying matter to targeted cells in a human body, including the steps of providing a predetermined amount of substance desired to be conveyed to the human body, embracing the substance with a predetermined amount of carrier to form a nano-size dosage form and administrating the nano-size dosage form to the human body. The carrier is capable of traveling around the human body through the nervous system of the human body along with the substance desired to be conveyed. The substance to be conveyed includes a wide range of substances such as medication, food supplements, or vitamins. The method conveys matters that are especially useful in curing illnesses or diseases. Even when used alone, the carrier helps curing illnesses or diseases.

### CROSS-REFERENCE OF RELATED APPLICATION

This is a non-provisional application of a provisional application having an application No. 60/605,678 and a filing date of Aug. 28, 2004.

### BACKGROUND OF THE PRESENT INVENTION

1. Field of Invention

The present invention relates to a method of conveying matter to targeted cells in a human body, and more particularly to a method of effectively conveying illness curing matter to cells in a human body, wherein the method comprising the steps of providing a predetermined amount of substance, embracing the substance with a predetermined amount of carrier to form a nano-size dosage form, wherein the nano-size dosage form is provided for traveling around the human body through the nervous system of the human body, and administrating the nano-size dosage form to the human body. Through the present invention, all substance administered to a user is fully absorbed by the targeted cells in a human body, such that toxic substance accumulation inside a human body is minimized and no excess drug is required.

2. Description of Related Arts

Conventional medication application methods include ingestions of tablets, capsules and solution, injection, inhalation and transdermal patch application. However, hundred percent drug absorption into the blood stream through these application methods can never be achieved, due to factors such as drug solubility, permeability, the ability of the patient's gastrointestinal tract and the patient's physiological status, resulting in excess drug intake which in turn accumulates in the liver causing damages to the liver.

Also, even when drugs are absorbed by body cells, they might necessarily be able to totally kill off disease causing viruses due to the fact that a virus is essentially nucleic acid molecules, which in turn are genomes, protected within strong protective shells. The genomes can enter body cells, replicate themselves at an extremely high rate, and encode proteins that are capable of forming protective shells around them. Not only are the strong protective shells making destruction of viruses very difficult, the highly mutative nature of viruses also make complete recovery from a viral disease impossible.

A classic example of viral disease is AIDS (Acquired immune deficiency syndrome) caused by caused by the human immunodeficiency virus (HIV), which continuously weakening the defense of the body by altering the immune system, causing infections to the body.

The elimination of viruses such as HIV is very difficult because, unlike bacteria which are microorganisms, viruses are simply strings of genes, which is not easily destroyed by drugs.

As a result, in order to improve drug dosage effectiveness and better eliminate viruses, a better mode of transporting illness curing matter into targeted cells in a human body must be provided.

### SUMMARY OF THE PRESENT INVENTION

A main object of the present invention is to provide a method of conveying matter to targeted cells in a human body, comprising the steps of providing a predetermined amount of substance, embracing the substance with a predetermined amount of carrier forming a nano-size dosage form, wherein the nano-size dosage form is provided for traveling around the human body through the nervous system of the human body, and administrating the nano-size dosage form to the human body.

Another object of the present invention is to provide a method of conveying matter to targeted cells in a human body, wherein the substance is conveyed by the carrier to cells in the human body through the body's nervous system, avoiding the possibilities that the drug looses its strength or function through the body's digestive system.

Another object of the present invention is to provide a method of conveying matter to targeted cells in a human body, wherein the substance is completely absorbed by targeted cells in the human body.

Another object of the present invention is to provide a method of conveying matter to targeted cells in a human body, wherein the carrier emits far infrared rays of a predetermined wavelength that kills disease-causing bacteria and viruses.

Another object of the present invention is to provide a dosage form for conveying a substance through the nervous system of a human body comprising a predetermined amount of nano-size substance, a predetermined amount of nano-size carrier for embracing the nano-size substance, wherein the nano-size carrier conveys the nano-size substance to targeted cells in the human body.

Another object of the present invention is to provide a dosage form, wherein the nano-size carrier is a naturally occurring substance and required by the human body, such that the nano-size carrier, which embraces the nano-size substance, not only is easily absorbed by the human body, reaching targeted cells more efficiently but also acts as a good supplement to the human body.

Another object of the present invention is to provide a dosage form, wherein the nano-size carrier can be orally administered or transdermally applied onto the skin of the human body.

Another object of the present invention is to provide a dosage form, wherein the nano-size carrier transmits information to bacteria and viruses in the human body, rearranging an amino acid sequence of the bacteria and viruses, changing the bacteria and viruses to proteins beneficial to the user.

Another object of the present invention is to provide a method of curing an illness, comprising the steps of providing a far infrared ray emitter, and administrating the far infrared ray emitter to a user.

Another object of the present invention is to provide a method of curing an illness, wherein the far infrared ray emitted by the far infrared ray emitter is provided for killing bacteria and viruses in the user.

Another object of the present invention is to provide a method of curing an illness, wherein instead of using drugs that would not only kill bacteria and viruses but are also harmful to a human body, the far infrared ray emitter is found in substances occurring in nature, which requires no complicated chemical processes such as extraction and synthesis, which would kill bacteria and viruses but will do no harm to the human body.

Another object of the present invention is to provide a method of curing an illness, wherein the far infrared ray emitted by the far infrared ray emitter can be customized, such that a predetermined wavelength of far infrared ray can be emitted according to the wavelength of the far infrared ray required to kill the bacteria and viruses inside the human body.

Another object of the present invention is to provide a method for producing a dosage form for conveying a substance through a nervous of a human body, comprising the steps of providing a predetermined amount of the substance, and embracing the substance with a carrier.

Another object of the present invention is to provide a method for producing a dosage form, wherein the carrier is capable of freely traveling into and out of the nervous system of the human body, such that the substance is conveyed along with the carrier to targeted cells in the human body. Another object of the present invention is to provide a method for producing a dosage form, wherein the carrier emits an infrared ray containing information to bacteria and viruses in the human body, rearranging an amino acid sequence of the bacteria and viruses, changing the bacteria and viruses to proteins beneficial to the user.

Accordingly, in order to accomplish the above objects, the present invention provides a method of conveying matter to targeted cells in a human body, comprising the steps of:
(a) providing a predetermined amount of substance to be conveyed;
(b) embracing the substance with a predetermined amount of carrier to form a nano-size dosage form; and
(c) administrating the nano-size dosage form to the human body, wherein the nano-size dosage form is provided for traveling around the human body through a nervous system of the human body, conveying the substance to the targeted cells.

The present invention has an alternative embodiment, wherein the present invention provides a dosage form for conveying a substance through a nervous system of a human body to targeted cells in a human body, comprising:
a predetermined amount of nano-size substance; and
a predetermined amount of nano-size carrier provided for traveling through the nervous system, wherein the nano-size carrier embraces the nano-size substance, such that the nano-size carrier travels through the nervous system along with the nano-size substance, bringing the nano-size substance to the targeted cells in the human body.

The present invention has yet another alternative embodiment, wherein the present invention provides a method of curing an illness, comprising the steps of:
(a) providing an illness curer; and
(b) conveying the illness curer to targeted cells in a human body through a nervous system of a human body.

The present invention has yet another alternative embodiment, wherein the present invention provides a method for producing a dosage form, wherein the dosage form is provided for conveying a substance through a nervous system of a human body, wherein the method comprises the steps of:
(a) providing a predetermined amount of the substance; and
(b) embracing the substance with a carrier.

These and other objectives, features, and advantages of the present invention will become apparent from the following detailed description, the accompanying drawings, and the appended claims.

### BRIEF DESCRIPTION OF THE DRAWINGS

FIG. 1 is a flow chart illustrating the method of conveying matters to cells in a human body according to the preferred embodiment of the present invention.

FIG. 2A illustrates a dosage form and its application according to an alternative embodiment of the present invention.

FIG. 2B illustrates an alternative dosage form and its application according to an alternative embodiment of the present invention.

FIG. 3A and FIG. 3B are flow charts illustrating a method of curing an illness according to another alternative embodiment of the present invention.

FIG. 4A and FIG. 4B are flow charts illustrating a method for producing a dosage form for conveying a substance through a nervous system of a human body according to another alternative embodiment of the present invention.

### DETAILED DESCRIPTION OF THE PREFERRED EMBODIMENT

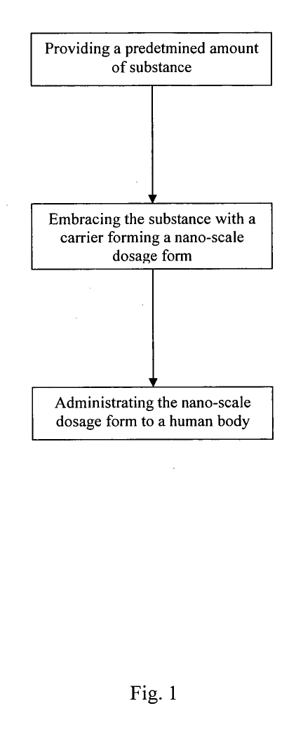

Referring to FIG. 1 of the drawings, a method of conveying matter to cells in a human body according to a preferred embodiment of the present invention is illustrated, wherein the method comprises the steps of providing a predetermined amount of substance, embracing the substance with a predetermined amount of carrier forming a nano-size dosage form, wherein the nano-size dosage form is provided for traveling around the human body through the nervous system of the human body, and administrating the nano-size dosage form to the human body.

When broken down to nano-size, the carrier is capable of freely traveling around the human body through the nervous system.

In order to embrace the substance with a carrier, the substance and the carrier are first mixed together to form a mixture. The mixture will then undergo a breaking down process, such that the mixture is broken down to nano-size. During the process, the nano-size carrier embraces with the nano-size substance, forming the nano-size dosage form, which is then administered to the human body.

Since this method is applicable to any substances, especially to any disease curing substances for curing any disease, the substance can be any medication needed. In the case of medication, the desired medication is first mixed with the carrier to form a mixture. The mixture is broken down to a nano-size dosage form, where the nano-size carrier embraces the nano-size medication.

Since the nano-size carrier is capable of traveling along the nervous system of the human body, the embraced nano-size medication will be brought to the targeted cells by the nano-size carrier through the nervous system of the human body.

Through this method, the amount of substance mixed with the carrier will be fully absorbed by the targeted cells. As a result, in the case where the substance is a medication, the amount of medication to be mixed with the carrier is wholly dependent on the amount of medication required by the body to cure a disease.

The ratio between the carrier and the substance mixed together is therefore adjustable. The ratio can vary between 1:1 and 1:0.02 by mole, depending on the amount of amount of substance required by the body. In the case of medication, the amount of medication to be mixed with the carrier is dependent on the amount of medication required to kill of the disease-causing bacteria or viruses.

This method is applicable to any substances to be transported to the cells of a human body, such as medication, health supplements or vitamins, such that absorption of such substances can be maximized.

The carrier can be any ions able to freely travel into and out of the nerve cells of the body's nervous system. Examples of such ions are calcium, sodium and magnesium ions, which can be either organic or inorganic. The reason is that such ions are naturally required by the body's nervous system to transmit electrical signals. With the help of such carriers, no harmful substances or chemicals are needed to carry the essential medication to targeted cells in the body.

According to the preferred embodiment of the present invention, the carrier is organic calcium, which comes from organic ceramics such as pearls and shells. Such calcium-containing organic ceramics are active substances that containing proteins. After mixing the substance with such organic ceramics, the mixture is broken down to nano-size by methods such as ultrasonic grinding, fluid energy grinding or ball-grinding.

As viruses are heavily negatively charged, the nerve cells serve as a highway for the viruses, such that the viruses can easily travel and infect the entire body. In the case of disease curing medication, since the carrier as described above is also capable of conveying the medication by means of the nervous system, not only can the amount of medication required to cure the disease be exactly the same as that applied, the medication can also be brought to the targeted cells more efficiently and accurately, as oppose to conventionally medication being transported to targeted cells by means of entering the blood vessels through the human body's digestive system.

Organic ceramics such as shells and pearls are capable of emitting far infrared rays, which can also enhance the bacteria and viruses killing. Under the influence of far infrared rays, and more particularly of far infrared rays with the wavelength of 0.02 μm, bacteria and viruses loose their reproduction ability, controlling and eventually killing off bacteria and viruses, including tough viruses such as the AIDS causing Human Immunodeficiency Virus (HIV).

In order to have the organic ceramics such as shells and pearls to emit far infrared rays of a predetermined wavelength, according to the preferred embodiment of the present invention, original far infrared rays existing inside such organic ceramics are cleared out by means of high voltage electrical field. After that, far infrared rays of a predetermined wavelength and amplitude are inputted into such organic ceramics by means of a high electrical voltage.

Organic ceramics such as shells and pearls also have the features of biochips. Biochips are biological molecules used for the processing of information. Information is inputted into the molecules of such organic ceramics by means of high electrical voltage.

Such organic ceramics can be use to transmit information altering the amino acid arrangement of bacteria and viruses in such a manner that no only will the bacteria and viruses be eliminated, they are also being transformed to proteins useful to the human body.

In the case of medication, since the medication is conveyed by means of the nervous system of the body, it can act upon bacteria and viruses existing in the nervous system, before such bacteria and viruses have the chance of reaching other body cells and successfully causing infections. Such method of conveying medication into cells of a human body makes curing of illnesses and diseases caused by bacteria and viruses at an earlier stage possible.

In an alternative embodiment of the method of conveying matter to cells in a human body, in order to embrace the substance with the carrier, the substance and the carrier are first broken down to form a nano-size substance and a nano-size carrier respectively.

The nano-size substance and the nano-size carrier are then mixed together. In order for the nano-size carrier to embrace the nano-size substance, a suitable embracing environment is to be provided, forming the nano-size dosage form.

By means of methods such as ultrasonic grinding, fluid energy grinding or ball-grinding, the substance and the carrier are both first broken down to nano-size before being mixed together. When the nano-size substance is mixed with the nano-size carrier, the carrier will not automatically embrace the substance. A suitable environment therefore has to be provided.

Providing a suitable embracing environment is essentially providing the embracing activation energy for the nano-size carrier to embrace the nano-size substance. According to this alternative embodiment, the embracing activation energy is provided by treating the mixed nano-size substance and nano-size carrier with ultrasound.

Under the ultrasound treatment, energy is provided to the nano-size carrier. Same as the preferred embodiment, the nano-size carrier, is ions able to freely travel into and out of the nerve cells of the human body's nervous system, such as calcium, sodium and magnesium ions. As a result, the ultrasonic energy activates the ions to an unstable energy level, such that the nano-size carrier embraces the nano-size substance.

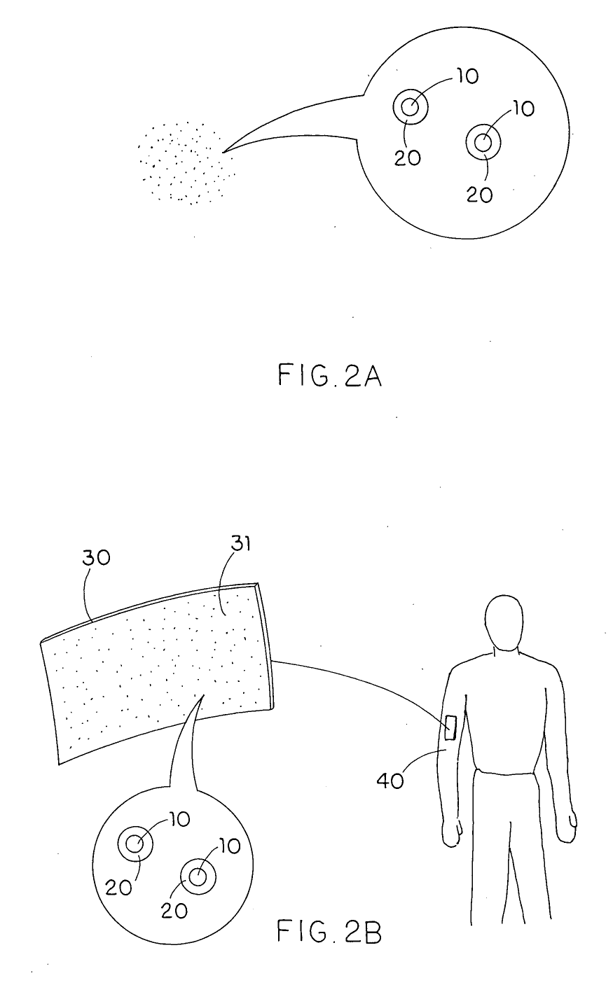

Referring to FIG. 2A of the drawings, an alternative embodiment of the present invention is illustrated, providing a dosage form for conveying a substance through a nervous system of a human body, wherein the dosage form comprises a predetermined amount of nano-size substance 10 and a predetermined amount of nano-size carrier 20 embracing the nano-size substance 10, wherein the nano-size carrier 20 is provided for conveying the nano-size substance 10 to targeted cells in the human body.

Conventionally, substances such as medication, food supplements and vitamins travel around the human body through the blood circulation system. Such substances enter the blood circulation system through the digestive system, injection and inhalation. Orally administered drugs must come in some dosage form of one way or another, such as solution, capsules, gels and tablets, depending on the desired drug releasing rate and the degree of resistance to digestion of the drug.

Under this embodiment of the present invention, the nano-size substance 10 can be any substances desired to be administered to the human body, such as medical drugs, herbal remedies, vitamins and other food supplements. When mixed with the nano-size carrier 20, the nano-size substance 10 is being embraced by the nano-size carrier 20.

According to this alternative embodiment, the nano-size carrier 20 comprises ions that are able to freely travel into and out of the nerve cells of the human body's nervous system. When the nano-size carrier 20 enters the human body, they can travel to the rest of the human body, through the human body's nervous system. Since the nano-size substance 10 is embraced by the nano-size carrier 20, the nano-size substance 10 reaches the entire body when the nano-size carrier 20 travels through the nervous system.

Examples of such ions are calcium, sodium and magnesium ions. Such ions are naturally found and required by the human body, especially by the nervous system of the human body. They are required either for the transmission of electrical signals through or maintaining the well-being of the nervous system.

As a result, upon being administered into the body, the nano-size carrier 20 can be quickly absorbed into the nervous system, and travels through the nervous system, reaching targeted cells efficiently.

The nano-size substances carrier 20 can be made of organic ceramics such as pearls or shells. As such organic ceramics are naturally occurring, and are safe to be administered into the body, without any potential health risk or dosage concerns. Such substances can be administered into the body orally or transdermally.

Referring to FIG. 2B of the drawings, the present alternative embodiment further comprises a patch body 30, which has an application surface 31, where the nano-size substance carrier 20, embracing the nano-size substance 10, is provided thereon. When applying the patch body 30 to a human body, the application surface 31 is attached onto a skin surface 40 of a human body, such that the nano-size carrier 20, along with the nano-size substance 10 is absorbed into the nervous system of the human body through the skin surface 40 of the human body.

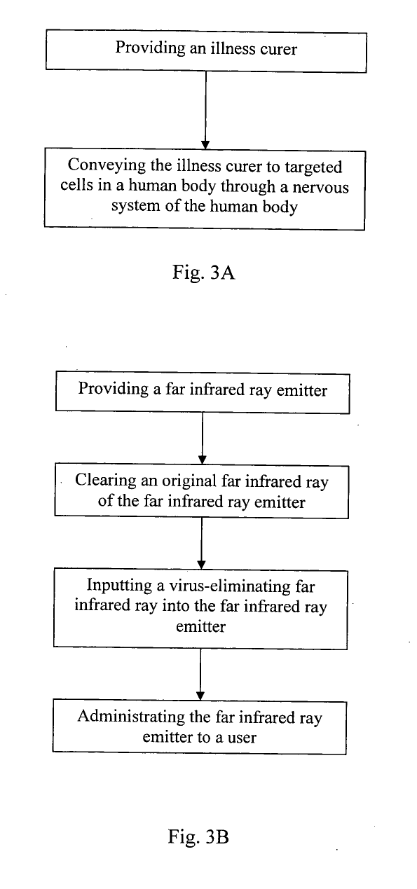

Referring to FIG. 3A of the drawings, an alternative embodiment of the present invention is illustrated, providing a method of curing an illness, comprising the steps of providing an illness curer, and conveying the illness curer to targeted cells in a human body, through a nervous system of the human body.

According to this alternative embodiment of the present invention, the illness curer is a far infrared ray emitter emitting a far infrared ray of a predetermined wavelength, wherein the far infrared ray contains biological information which is capable of altering an amino acid arrangement of the targeted cells.

Disease causing viruses are difficult to be eliminated by drugs, because unlike bacteria, viruses are not strictly living cells, but are genomes, protected within strong protective shells. Such strong protective shells are very resistive to toxic substances released by drugs, and, as a result, protecting genomes inside the strong protective shells. Viruses affect a human body by interfering with the electrical signal transmission of the nervous system of the human body, as viruses themselves also express themselves by means of electrical pulse.

As a result, their express of information, as well as the viruses themselves, can be destructed by the use of far infrared ray. With far infrared ray with the wavelength of 0.02 μm, viruses will loose their reproductive ability, as well as their ability to communicate through electrical pulse, and yet the body cells of the human body will not be affected. Also, the far infrared rays also transform the proteins of the viruses to proteins useful to the human body.

According to this alternative embodiment of the present invention, the far infrared ray emitter is ceramics, and more particularly nano-size organic ceramics such as pearls and shells.

Prior to any treatments, such nano-size organic ceramics emit original far infrared rays with their own wavelengths. Referring to FIG. 3B, in order for them to emit virus-eliminating far infrared rays of a predetermined wavelength, the original far infrared rays must first be cleared out from the nano-size organic ceramics. Then, the virus-eliminating far infrared rays are inputted into the nano-size organic ceramics.

The original far infrared rays are cleared out by means of a high voltage electrical field. The virus-eliminating far infrared rays are inputted into such nano-size organic ceramics by means of a high electrical voltage.

In order to enhance the illness curing effectiveness, a nano-size substance such as medication and health supplements is embraced by the nano-size organic ceramics, such that the nano-size organic ceramics is conveyed to targeted cells in the human body through the nervous system of the human body along with the nano-size substance.

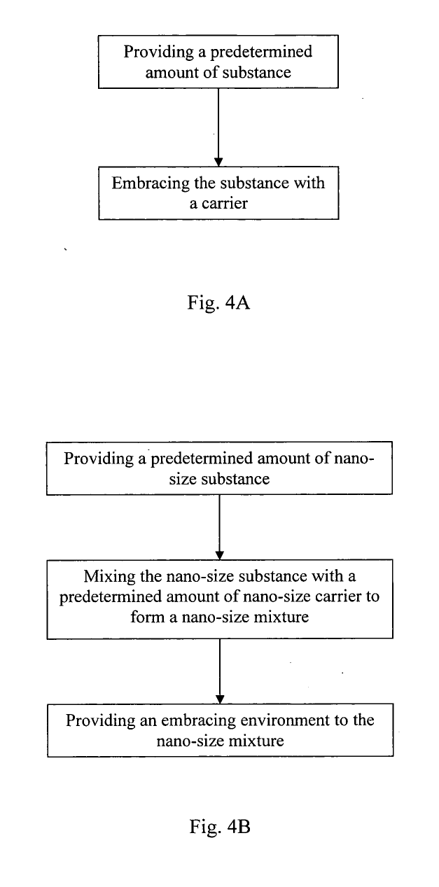

Referring to FIG. 4A of the drawings, another alternative embodiment of the present invention is illustrated, providing a method for producing a dosage form for conveying a substance through a nervous system of a human body, wherein the method comprises the steps of providing a predetermined amount of substance, and embracing the substance with a carrier.

In order for the carrier to embrace the substance, the carrier and the substance are first mixed together to form a mixture. The mixture will then go through a breaking down process, such that the mixture is broken down to nano-size. The breaking down process provides the necessary activation energy for the carrier to embrace the substance.

The substance can be anything that is desired to be transported to the cells of a human body, such as medication, health supplements or vitamins. The carrier can be any ions able to freely travel into and out of the nerve cells of the body's nervous system. Examples of such ions are calcium, sodium and magnesium ions, which can either be organic or inorganic. Such ions are naturally required by the body's nervous system to transmit electrical signals. The carrier is provided for carrying the substance to targeted cells in the human body, such that no other harmful substances or chemicals are needed.

According to this alternative embodiment of the present invention, the carrier is organic calcium coming from organic ceramics such as pearls and shells. As a result, the substance, which is desired to be transported to the cells of the human body, is mixed with the pearls or shells.

The ratio in which the substance is mixed with the carrier is wholly dependent on the amount of substance that wished to be transported to the cells of the human body because, through this method, the amount of substance administered to the human body will be totally absorbed by the targeted cells of the human body. The ratio between the carrier and the substance varies between 1:1 and 1:0.02 by mole. The ratio is such that the entire amount of substance is embraced by the carrier.

In order to ensure that the dosage form is up to health standard and free of contaminants, before the step of mixing the substance with a carrier to form a mixture, there is a step of disinfecting the substance and the carrier. Disinfection can be achieved by processes such as treating the substance and the carrier with ultraviolet rays or chlorination.

The breaking down of the mixture to nano-size can be achieved by processes such as ultrasonic grinding, fluid energy grinding or ball-grinding.

Alternatively, according to FIG. 4B of the drawings, the substance provided is a predetermined amount of nano-size substance and the carrier is a predetermined amount of nano-size carrier. After mixing the nano-size substance with the nano-size carrier to form a nano-size mixture, in order for the nano-size carrier to embrace the nano-size substance, an embracing environment is to be provided to the nano-size mixture.

By providing the embracing environment, an embracing activation energy required by the nano-size carrier to embrace the nano-size substance is provided. According to this alternative embodiment, the embracing activation energy is provided by treating the nano-size mixture with ultrasound.

Under the ultrasound treatment, the embracing activation required by the nano-size carrier to embrace the nano-size substances is provided. As the nano-size carrier is ions able to freely into and out of the nerve cells of the human body's nervous system, such as calcium, sodium and magnesium ions. As a result, the ions are activated by the ultrasound to an unstable energy level. In order for the nano-size carrier to be stable again, the nano-size carrier embraces the nano-size substance, thereby returning to a stable energy level.

The present invention according to this alternative embodiment further comprises a step of inputting far infrared rays of a predetermined wavelength and amplitude to the nano-size carrier, in such a manner that the nano-size carrier emits the far infrared rays in the human body, so as to enhance the virus-eliminating performance of the dosage form.

Before the step of inputting far infrared rays to the nano-size carrier, there further is a step of clearing out original far infrared rays present in the nano-size carrier. The clearing out of original far infrared rays is achieved by applying a high voltage electrical field to the nano-size carrier.

As organic ceramics such as shells and pearls also display characteristics of biochips, information can be inputted into and transmitted by the molecules of the organic ceramics for altering the amino acid arrangement of bacteria and viruses. As a result, this alternative embodiment of the present invention further has a step of inputting information into the nano-size carrier. The inputting of information into the nano-size carrier is achieved by the application of high electrical voltage.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of conveying matter to targeted cells in a human body, comprising the steps of:
(a) providing a predetermined amount of substance to be conveyed, and a predetermined amount of carrier having carrying ions, wherein said carrying ions of said carrier are adapted for traveling around said human body through a nervous system thereof;
(b) embracing said substance with said carrier to form a nano-size dosage form; and
(c) administrating said nano-size dosage form to said human body, wherein said carrying ions of said carrier are adapted to carry said substance in said human body to convey said substance to said targeted cells.

2. The method as recited in claim 1, in step (a), further comprising a step of:
(a.1) storing far infrared signal into said carrier, wherein said far infrared signal contains biological information of said targeted cells that said substance to be conveyed.

3. The method as recited in claim 2, in step (a.1), further comprising the steps of:
(a.11) clearing out an original far infrared signal of said carrier by applying a high voltage electrical field to said carrier; and
(a.12) inputting said far infrared signal into said carrier by applying a high electrical voltage.

4. The method, as recited in claim 2, wherein a wavelength of said far infrared signal is 0.02 μm for altering an amino acid arrangement of said targeted cells.

5. The method, as recited in claim 3, wherein a wavelength of said far infrared signal is 0.02 μm for altering an amino acid arrangement of said targeted cells.

6. The method, as recited in claim 1, wherein said carrying ions have positive charge adapted for traveling to said targeted cells having negative charge.

7. The method, as recited in claim 5, wherein said carrying ions have positive charge adapted for traveling to said targeted cells having negative charge.

8. The method as recited in claim 1, in step (b), further comprising the steps of:
(b.1) mixing said substance with said carrier to form a mixture;
(b.2) breaking down said mixture into nano-size; and
(b.3) treating said mixture with ultrasound for enhancing activation of said carrying ions of said carrier.

9. The method as recited in claim 5, in step (b), further comprising the steps of:
(b.1) mixing said substance with said carrier to form a mixture;
(b.2) breaking down said mixture into nano-size; and
(b.3) treating said mixture with ultrasound for enhancing activation of said carrying ions of said carrier.

10. The method as recited in claim 7, in step (b), further comprising the steps of:
(b.1) mixing said substance with said carrier to form a mixture;
(b.2) breaking down said mixture into nano-size; and
(b.3) treating said mixture with ultrasound for enhancing activation of said carrying ions of said carrier.

11. An illness curer adapted for conveying to targeted cells of a human body through a nervous system thereof, comprising:
a predetermined amount of substance for being conveyed to said targeted cells to cure said targeted cells by said substance; and
a predetermined amount of carrier having carrying ions adapted for traveling around said human body through said nervous system thereof, wherein said substance is embraced with said carrier and is formed in a nano-size dosage in such a manner that said carrying ions of said carrier carry said substance in said human body to convey said substance to said targeted cells when said nano-size dosage is administrated to said human body.

12. The illness curer, as recited in claim 11, wherein said carrier comprises a far infrared emitter generating far infrared signal which contains biological information of said targeted cells that said substance to be conveyed.

13. The illness curer, as recited in claim 11, wherein a wavelength of said far infrared signal is 0.02 μm for altering an amino acid arrangement of said targeted cells.

14. The illness curer, as recited in claim 12, wherein a wavelength of said far infrared signal is 0.02 μm for altering an amino acid arrangement of said targeted cells.

15. The illness curer, as recited in claim 14, wherein said carrier is selected from a group consisting of organic calcium ions, inorganic calcium ions, organic sodium ions, inorganic sodium ions, organic magnesium ions and inorganic magnesium ions.

16. The illness curer, as recited in claim 14, wherein said carrier comprises organic ceramics having organic calcium ions as said carrying ions.

17. The illness curer, as recited in claim 15, wherein said carrying ions have positive charge adapted for traveling to said targeted cells having negative charge.

18. The illness curer, as recited in claim 16, wherein said carrying ions have positive charge adapted for traveling to said targeted cells having negative charge.

19. The illness curer, as recited in claim 19, further comprising a patch body having an application surface, wherein said nano-size dosage is provided on said application surface for applying on a skin of said human body so as to convey said substance through said skin.

20. The illness curer, as recited in claim 20, further comprising a patch body having an application surface, wherein said nano-size dosage is provided on said application surface for applying on a skin of said human body so as to convey said substance through said skin.
